(19) United States
(12) Patent Application Publication
(10) Pub. No.: US 2024/0269386 A1
(43) Pub. Date: Aug. 15, 2024

(54) REUSABLE INJECTION APPARATUS AND METHOD

(71) Applicant: Purdue Research Foundation, West Lafayette, IN (US)

(72) Inventors: Nathan B. French, Naperville, IL (US); Deepa Jayasankar, Carmel, IN (US); Brianna M. Shannon, Bethel Park, PA (US); Victoria C. Sinfield, West Lafayette, IN (US); Nickolaus G. Will, Valparaiso, IN (US)

(73) Assignee: Purdue Research Foundation, West Lafayette, IN (US)

(21) Appl. No.: 18/434,440

(22) Filed: Feb. 6, 2024

Related U.S. Application Data
(60) Provisional application No. 63/444,545, filed on Feb. 9, 2023.

(51) Int. Cl. A61M 5/20

(52) U.S. Cl. CPC A61M 5/2033; A61M 2005/2026; A61M 2005/206; A61M 2005/2073; A61M 2205/121

(57) ABSTRACT

An auto-injector includes a housing and an actuation assembly. The housing includes a central cavity, opposing ends, and a longitudinal axis defined spanning between the opposing ends. The actuation assembly is shaped to position within the central cavity. The actuation assembly is configured to at least partially internally house a spring, a spring compression member, a needle operable to eject a fluid therefrom, a firing member selectively translatable between an unfired position and a fired position, and a firing initiation member. While in the unfired position, the needle is held within the central cavity of the housing. While in the fired position, the needle is extended outward from the central cavity of the housing.

### CROSS-REFERENCE TO RELATED APPLICATIONS

This application is related to and claims the priority benefit of U.S. Provisional Patent Application No. 63/444,545, entitled “Reusable Injection Apparatus and Method,” filed Feb. 9, 2023, the contents of which are hereby incorporated by reference in their entirety into the present disclosure.

### TECHNICAL FIELD

The invention relates to medical devices, and specifically to a reusable and resettable injector or auto-injector for delivering medicament to an injection site, and methods of use thereof.

### BACKGROUND

This section introduces aspects that may help facilitate a better understanding of the disclosure. Accordingly, these statements are to be read in this light and are not to be understood as admissions about what is or is not prior art.

An automatic injector or auto-injector is a device designed to allow a user to self-administer a pre-measured dose of a medicament composition subcutaneously or intramuscularly, usually in an emergency situation. Automatic injectors are used, for example, to treat anaphylactic (i.e., severe allergic) reactions and to administer antidotes for certain poisons, such as chemical nerve agents and various drug compositions such as diazepam.

A typical auto-injector has a housing, inside of which is a cartridge. The cartridge has one or several chambers containing medicament compositions or components thereof and is adapted to be attached to a needle assembly. The cartridge can hold either a pre-mixed liquid medicament or a solid medicament and a liquid that are mixed prior to injection. The housing carries an actuation assembly with a stored energy source, for example, a compressed spring. Activation of the actuation assembly causes a sequence of movements, whereby the needle extends from the auto-injector into the user so that the medicament compound is then forced through the needle and into the user. After delivery of the dose of medicament into the injection site, the needle remains in an extended position. If the auto-injector is of the type designed to carry plural components of the medicament composition in separate, sealed compartments, structure may be included that forces the components to mix when the actuation assembly is activated.

An anaphylactic allergy is when the body's immune response reacts to a substance such as an insect sting or various foods. Allergies cannot be cured and the only treatment for an anaphylactic reaction is epinephrine. If epinephrine is not administered within 15 minutes, anaphylactic reactions can be fatal. This is because it causes vasoconstriction, increased blood pressure, and increased heart rate via α1-adrenergic receptors, β1-adrenergic receptors, and β2-adrenergic receptors. In the United States, approximately six million people suffer from anaphylactic allergies annually, and approximately 3.6 million of those people are not readily equipped with an epinephrine auto-injector (EAI) to treat the allergic reaction. Additionally, approximately 56% of patients are concerned about the cost of EAIs, and approximately 36% of patients are unable to afford one. Further, existing EAIs are single-use devices, meaning the entire mechanism must be replaced if used or when the epinephrine expires, typically within a year. Therefore, an improved injection apparatus is needed to reduce cost by making the apparatus reusable and the cartridge replaceable.

### SUMMARY

The present disclosure describes a reusable and resettable injector or auto-injector for delivering medicament to an injection site. Particularly, a spent medicament cartridge from the auto-injector may be removed and replaced, and the auto-injector may be reset and prepared for an additional use. In some embodiments, the auto-injector, can include a housing and an actuation assembly. The housing can have a central cavity, opposing ends, and a longitudinal axis defined spanning between opposing ends. The housing can include at least one first protrusion extending toward the central cavity from an inward facing surface of the housing. The actuation assembly can be shaped to position within the central cavity of the housing, and can define at least one first slot accessible from an interior surface of the actuation assembly and at least one second slot accessible from an exterior surface of the actuation assembly. The at least one first protrusion of the housing is can be configured to align with and translate within the at least one second slot.

Further, the actuation assembly can at least partially internally house a compression spring, a spring compression member, a need, and a firing member. The spring compression member can be configured to couple with the actuation assembly and contact the compression spring, and the spring compression member can include at least one second protrusion configured to translate within the at least one first slot of the actuation assembly. The needle can be selectively operable to eject a fluid therefrom, and the needle can be selectively operable to extend from a proximal end of the housing. The firing member can be selectively translatable between an unfired position and a fired position. While in the unfired position, the compression spring can be restricted into a compressed state, whereby the compression spring in the compressed state can be operable to position the needle within the central cavity of the housing. While in the fired position, the compression spring can be unrestricted into an expanded state, whereby the compression spring in the expanded state can be operable to transition the needle outward from the central cavity of the housing.

In some embodiments, the auto-injector can include a firing initiation member configured to couple between the spring compression member and the actuation assembly and translate parallel to the longitudinal axis between a first position and a second position relative to the housing. In the first position, the firing member can be held in the unfired position. During a translation into the second position, the firing member can be translated into the fired position.

In other embodiments, the actuation assembly can include a raised ridge around the internal surface. The firing member can define a cylindrical shape having a first radius at a first end and a second radius at a second end, with the second radius being larger than the first radius. Additionally, the second end of the firing member can be formed by a plurality of legs. Each leg of the plurality of legs can be shaped to lodge against the raised ridge while the firing member is in the unfired position. In additional embodiments, the firing initiation member can be configured to translate over each of the plurality of legs to thereby decrease the second radius of the firing member and dislodge each leg of the plurality of legs from the raised ridge.

In some embodiments, resetting an auto-injector can include one or more steps. In one aspect, such a method can include inserting an unspent syringe into the syringe holder, applying a distal force to the syringe holder thereby driving the firing member distally, and positioning the firing member into the unfired position. In the unfired position, each leg of the plurality of legs can be lodged against the raised ridge of the actuation assembly.

This summary is provided to introduce a selection of the concepts that are described in further detail in the detailed description and drawings contained herein. This summary is not intended to identify any primary or essential features of the claimed subject matter. Some or all of the described features may be present in the corresponding independent or dependent claims, but should not be construed to be a limitation unless expressly recited in a particular claim. Each embodiment described herein does not necessarily address every object described herein, and each embodiment does not necessarily include each feature described. Other forms, embodiments, objects, advantages, benefits, features, and aspects of the present disclosure will become apparent to one of skill in the art from the detailed description and drawings contained herein. Moreover, the various apparatuses and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts an isometric view of one exemplary injection apparatus;

FIG. 2 depicts an exploded view of the injection apparatus of FIG. 1;

FIG. 3 depicts a cross-sectional view of the injection apparatus of FIG. 1, taken along cutting line A-A of FIG. 1;

FIG. 4A depicts an isometric view of the main shaft component of the apparatus of FIG. 1;

FIG. 4B depicts a side view of the main shaft component of FIG. 4A;

FIG. 4C depicts a top plan view of the main shaft component of FIG. 4A;

FIG. 5A depicts an isometric view of the outer shroud component of the apparatus of FIG. 1;

FIG. 5B depicts a side view of the outer shroud component of FIG. 5A;

FIG. 5C depicts a bottom plan view of the outer shroud component of FIG. 5A;

FIG. 6A depicts an isometric view of the spring compression member of the apparatus of FIG. 1;

FIG. 6B depicts a side view of the spring compression member of FIG. 6A;

FIG. 6C depicts a bottom plan view of the spring compression member of FIG. 6A;

FIG. 7A depicts an isometric view of the initiation member of the apparatus of FIG. 1;

FIG. 7B depicts a side view of the initiation member of FIG. 7A;

FIG. 7C depicts a top plan view of the initiation member of FIG. 7A;

FIG. 8A depicts an isometric view of the firing member of the apparatus of FIG. 1;

FIG. 8B depicts a side view of the firing member of FIG. 8A;

FIG. 9A depicts an isometric view of the syringe holder component of the apparatus of FIG. 1;

FIG. 9B depicts a side view of the syringe holder component of FIG. 9A;

FIG. 9C depicts a bottom plan view of the syringe holder component of FIG. 9A;

FIG. 10A depicts an isometric view of the safety clip component of the apparatus of FIG. 1;

FIG. 10B depicts a side view of the safety clip component of FIG. 10A;

FIG. 10C depicts a top plan view of the safety clip component of FIG. 10A;

FIG. 11A depicts an isometric view of the cap component of the apparatus of FIG. 1;

FIG. 11B depicts a side view of the cap component of FIG. 11A;

FIG. 11C depicts a top plan view of the cap component of FIG. 11A;

FIG. 12 depicts a perspective view of one exemplary method of replacing a syringe from and resetting the apparatus of FIG. 1 for additional uses, showing one exemplary apparatus stand in use;

FIG. 13A depicts an isometric view of an apparatus stand used for replacing a syringe from and resetting the apparatus of FIG. 1;

FIG. 13B depicts a side view of the apparatus stand of FIG. 13A; and

FIG. 13C depicts a bottom plan view of the apparatus stand of FIG. 13A.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown, or the precise experimental arrangements used to arrive at the various graphical results shown in the drawings.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Existing epinephrine auto-injectors are typically only capable of administering one dose of epinephrine before becoming unusable, or spent, and users often do not carry extra epinephrine auto-injectors (e.g., EpiPens) with them. Additionally, only 0.3 mL of the epinephrine-containing solution is administered for an effective dose, while 1-2 mL of the solution remains in the auto-injector un-used once the auto-injector is spent. Some existing auto-injector designs, such as the EPIPEN Twinject, allow for the administration of a second dose but require dismantling of the mechanism and manual administration of a 0.15- or 0.30-mL dose, which decreases the usability of the device. Additionally, 56% of patients are concerned with cost with 36% of patients being unable to afford even one such device.

Accordingly, described herein is an injection apparatus which addresses the clinical need for a reusable, low-cost epinephrine auto-injector. While the apparatus and method describe epinephrine, it should be understood that clinical injectors and auto-injectors provide a much wider range of applicability for administering various treatments, thus the apparatus and method described is not intended to be limited to sole use of administering epinephrine.

Particularly, the spring-loaded injection design described allows a firing spring to be re-compressed for additional uses, and a removable cap allows access to the drug vial (e.g., the syringe) and needle for easy replacement.

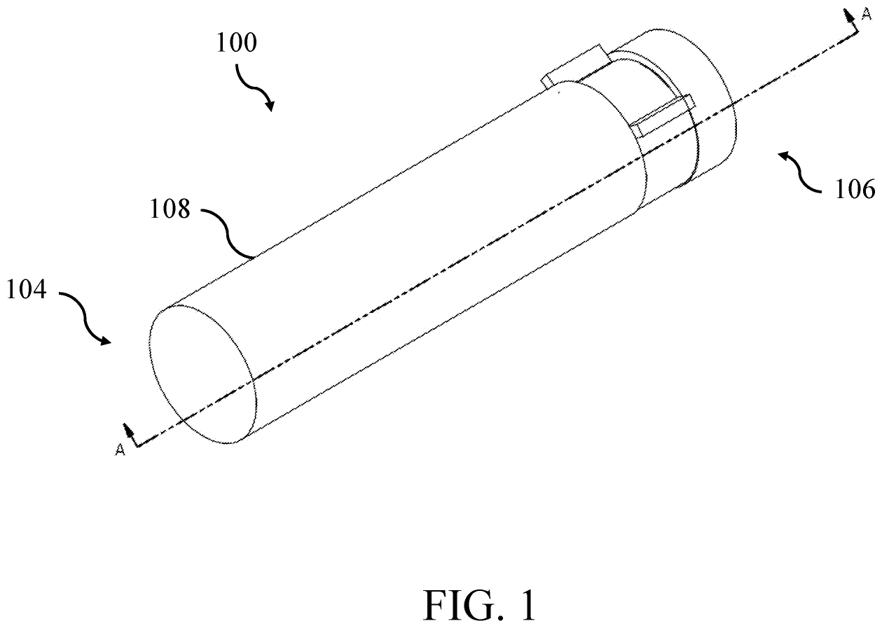

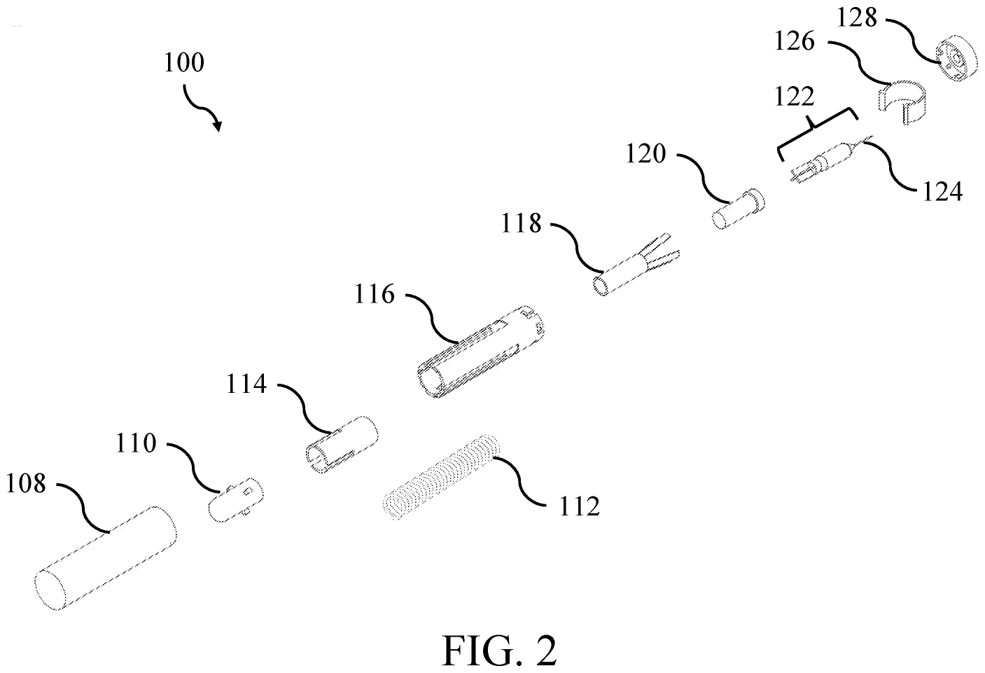

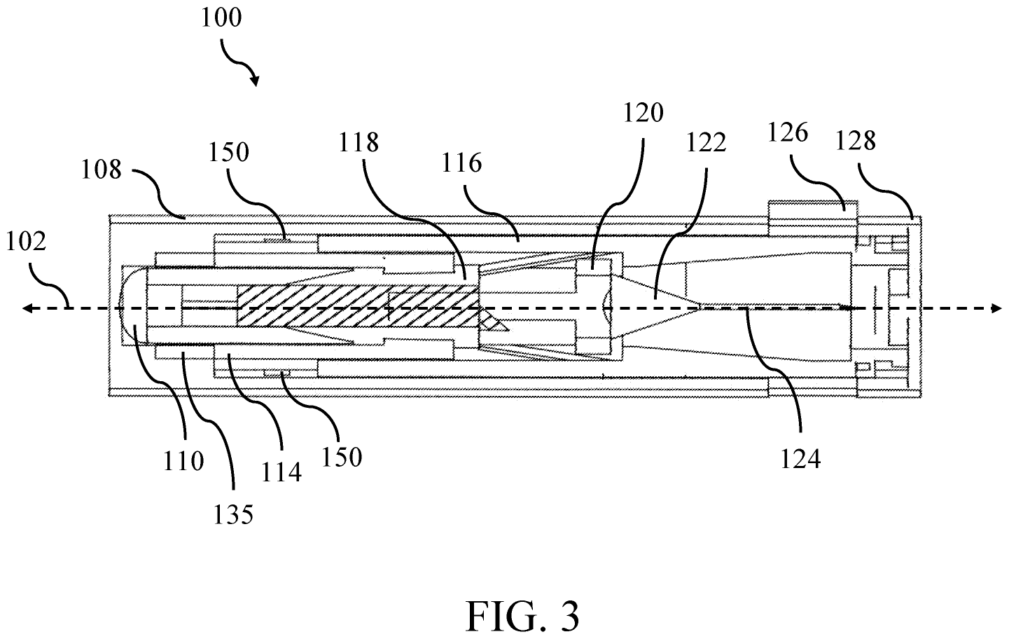

FIG. 1 shows one assembled embodiment of an injection apparatus (100) which is refillable and resettable, and therefore reusable to inject a plurality of treatments. As will be described in greater detail below, the apparatus includes a generally cylindrical shape defining a central longitudinal axis (102) (see, FIG. 3), where a distal end (104) is configured to be axially depressed by a user to release a syringe needle (see, FIG. 2) from the opposing proximal end (106). The apparatus (100) is therefore sized to fit within a single human palm such that a user can apply pressure to the distal end (104) with one hand or finger to release the needle from the proximal end (106) that is positioned in contact with a human body. In some applications, a user may grip the outer shroud (108) with all fingers curled around the outer shroud (108) to apply the proximal force necessary to release the syringe needle.

FIG. 2 shows the apparatus (100) disassembled, and FIG. 3 shows a cross-sectional view of the apparatus (100) assembled and in an unfired position (i.e., the needle has not been extended to administer a treatment). As will be described in greater detail below, and with continued reference to FIGS. 2-3, the apparatus generally includes a plurality of individual parts, such as the outer housing or shroud (108), the spring compression member (110), the spring (112), the initiation member (114), the actuation assembly or main shaft (116), the firing member (118), the syringe holder (120), the syringe (122) (which includes the needle (124)), the safety clip (126), and the cap (128).

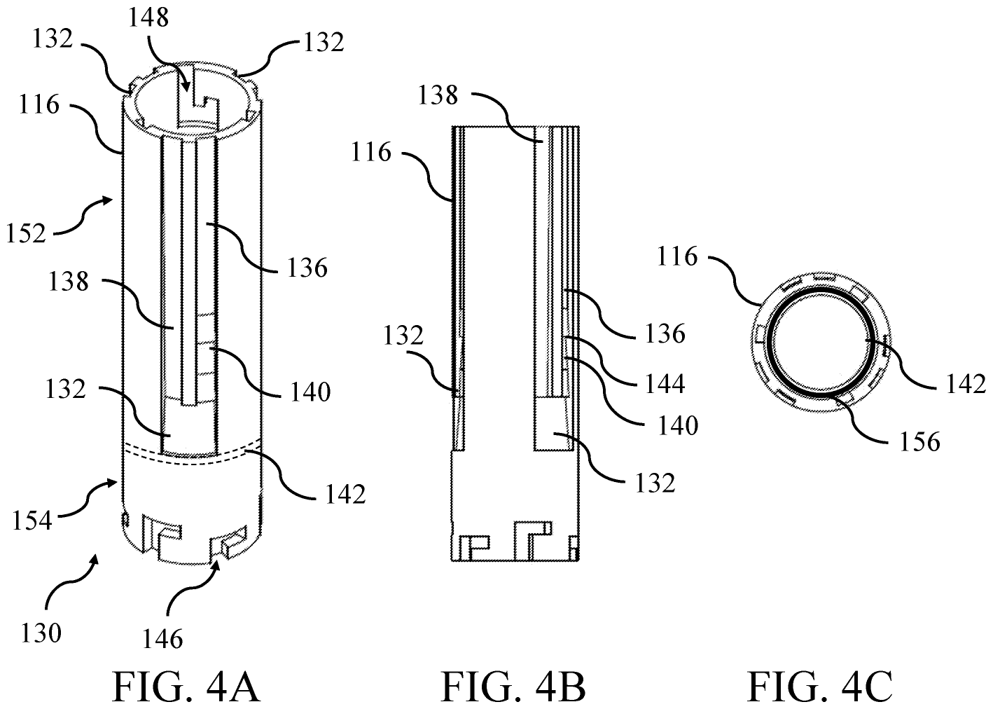

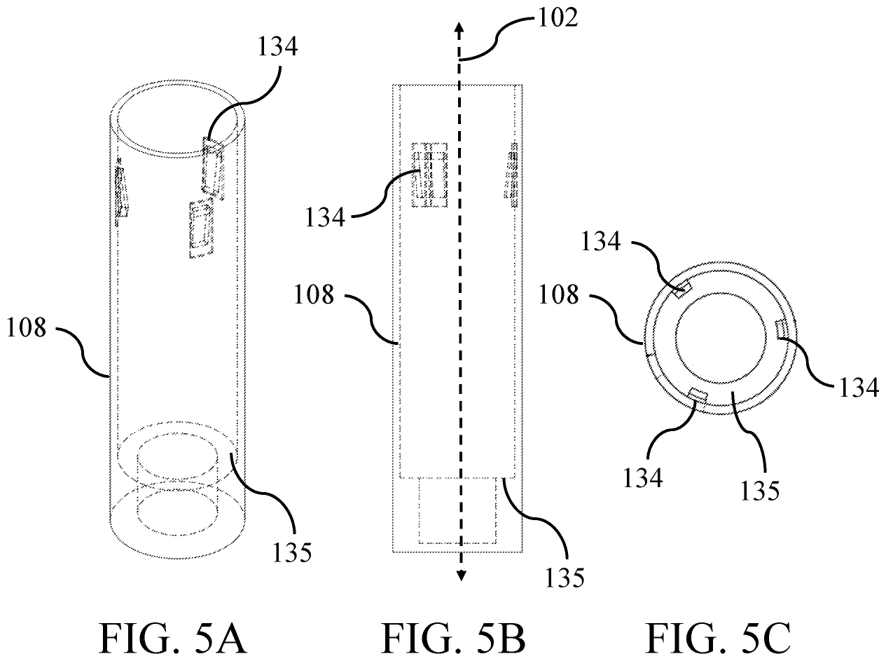

FIGS. 4A-4C show an exemplary embodiment of the main shaft (116). The main shaft (116) is configured to house several other components within it, such as the spring compression member (110), the initiation member (114), the firing member (118), the spring (112), the syringe holder (120), and the syringe (122). The needle (124) of the syringe (122) is configured to extend outward from the proximal (i.e., skin-facing) end (130). The main shaft (116) is generally cylindrical such that it slides into the outer shroud (108) while making contact around its circumference with the outer shroud (108). The main shaft (116) defines a plurality of slots both on its interior facing surface and its exterior facing surface. Particularly, the main shaft (116) includes U-shaped slots (132) on its exterior facing surface that is configured to interact with protrusions (134) (see, FIGS. 5A and 5C) located on the inner facing surface of the outer shroud (108). The U-shaped slot (132) includes a first leg (136) and a second leg (138) coupled together at the proximal end (130). In an unfired position, the protrusions (134) (see, FIGS. 5 and 5C) of the outer shroud (108) are positioned within a groove (140) of the first leg (136) such that an edge (144) defined by the groove (140) prevents the main shaft (116) and outer shroud (108) from being uncoupled. After a firing (i.e., an extension of the needle away from the main shaft (116) to administer a treatment), the protrusions (134) of the outer shroud (108) can be moved from the first leg (136) to the second leg (138) such that the outer shroud (108) and main shaft (116) can be uncoupled.

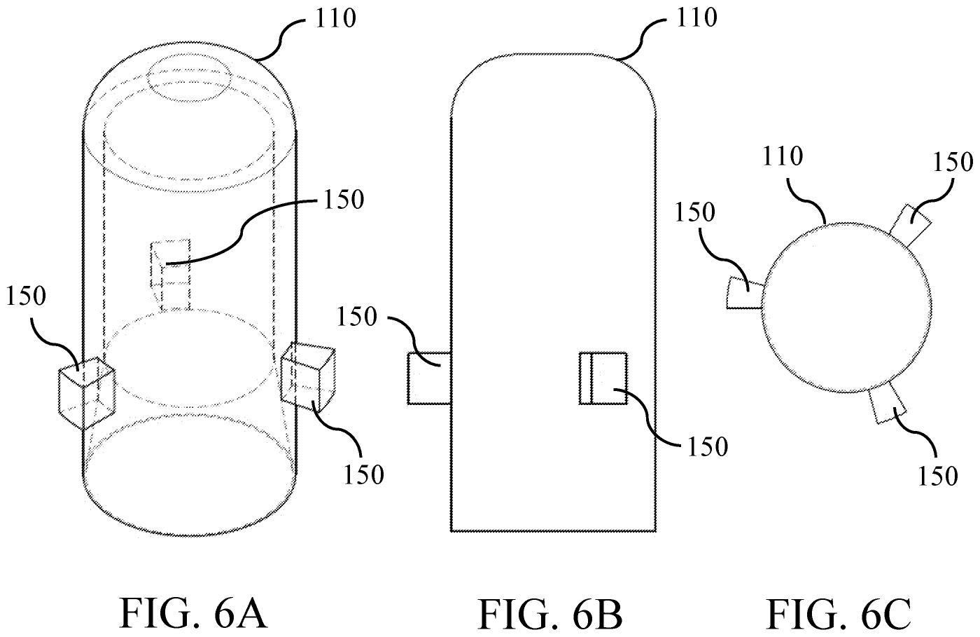

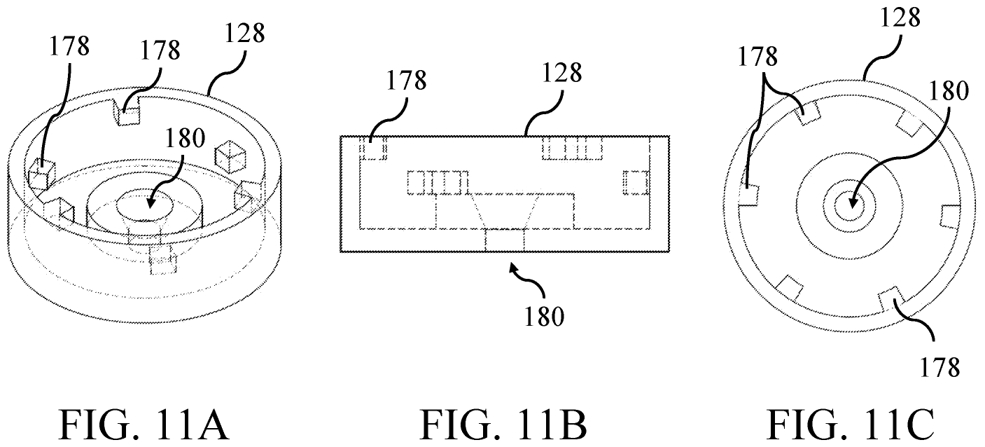

The main shaft (116) further includes a plurality of proximal slots (146) whereby the cap (128) (see, FIGS. 11A-11C) is configured to couple with the main shaft (116), as will be described below. Still further, the main shaft (116) includes a plurality of distal slots (148) whereby the protrusions (150) of the spring compression member (110) (see, FIGS. 6A-6C) may be inserted to affix the spring compression member (110) to the main shaft (116), as will be described.

Additionally, the inner diameter of the main shaft (116) defines a distal section (152) having a first diameter and a proximal section (154) having a second diameter, the first diameter being slightly larger than the second diameter (that is, the proximal section (154) of the main shaft (116) has a smaller inner diameter than the distal section (152) of the main shaft (116)). Particularly, the transition from the distal section (152) to the proximal section (154) defines a ridge (142). The ridge (142) can optionally include a washer (156) positioned on the ridge (142).

FIGS. 5A-5C show an exemplary embodiment of the outer shroud (108). The outer shroud (108) is configured to protect the inner components and to provide as a rigid grip for a user to administer the treatment from within. As such, the outer shroud (108) defines a cylindrical shape as its exterior surface and includes a plurality of protrusions (134) on its inner surface that are configured to correspond with the U-shaped slot (132) as described above, along with one or more protrusions configured to contact the initiation member (114), as will be described. As shown in FIG. 5C, the outer shroud includes an inner lip (135) protruding inward from the interior surface such that at the position of the lip (135) the outer shroud (108) defines an inner diameter greater than the outer surface diameter of the spring compression member (110), such that the initiation member (114) sits within the lip (135) which allows the outer shroud (108) to contact and translate the initiation member (114) longitudinally as a user grips and applies proximal pressure (i.e., sliding the outer shroud (108) in a direction toward the needle and skin surface for puncturing with the needle). In some embodiments, the outer shroud (108) can include a temperature sensitive label (not shown) on an exterior surface that indicates an apparatus (100) temperature history for clinical safety.

FIGS. 6A-6C show an exemplary embodiment of the spring compression member (110). The spring compression member (110) is configured to be positioned adjacent the distal end (152) of the main shaft (116) by way of the protrusions (150) coupling within the plurality of distal slots (148) of the main shaft (116). As such, the spring compression member (110) and the main shaft (116) are configured to hold a fixed position relative to each other during and before use of the apparatus (100). Once the spring compression member (110) is in place, it is configured to abut the distal end of the spring (112) that is held compressed within the apparatus (100).

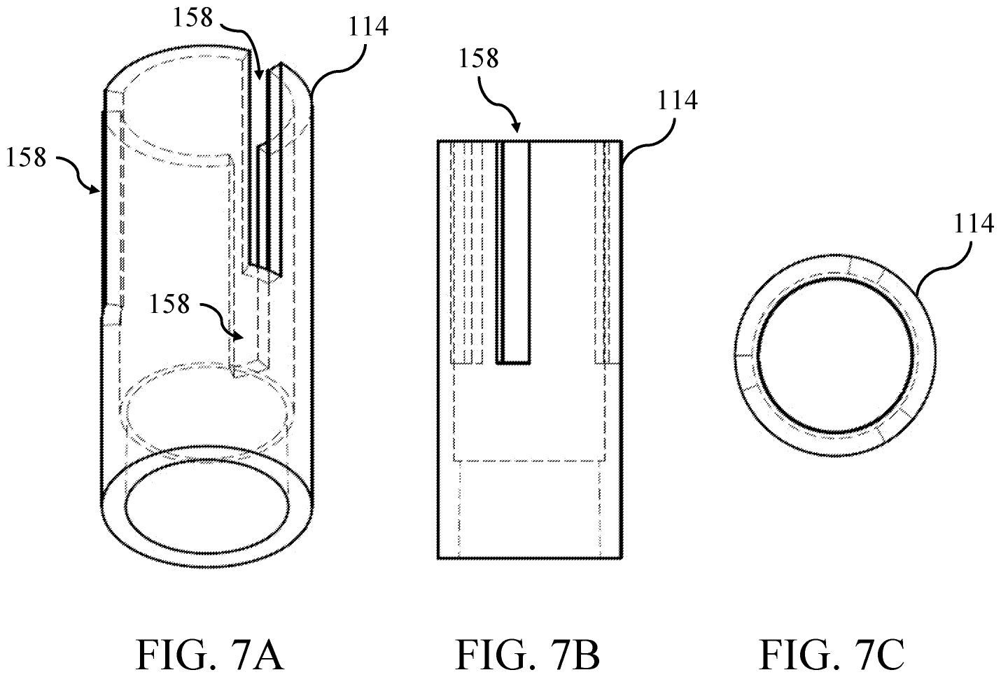

FIGS. 7A-7C show an exemplary embodiment of the initiation member (114). The initiation member (114) is configured to position between the main shaft (116) and the spring compression member (110). Moreover, the initiation member (114) includes a plurality of slots (158) configured to accept the protrusions (150) of the spring compression member (110) therein. As such, the initiation member (114) is configured to translate parallel to or along the longitudinal axis (102) relative to both the main shaft (116) and the spring compression member (110) as result of the proximal force of the outer shroud (108), the longitudinal movement directed by the one or more protrusions (134) on the distal interior surface of the outer shroud (108) forcing the initiation member (114) proximally as user applies proximal pressure to the outer shroud (108) during a firing, until the initiation member (114) meets the ridge (142) on the main shaft (116). Particularly, to initiate a firing of the needle (124), the outer shroud (108) and therefore the initiation member (114) are axially translated by the user in a proximal direction relative to the main shaft (116). As will be described below, the proximal translation of the initiation member (114) allows the compressed spring (112) to release and expand and press proximally against the syringe (122) to extend the needle (124) outward from the main shaft (116) through the cap (128).

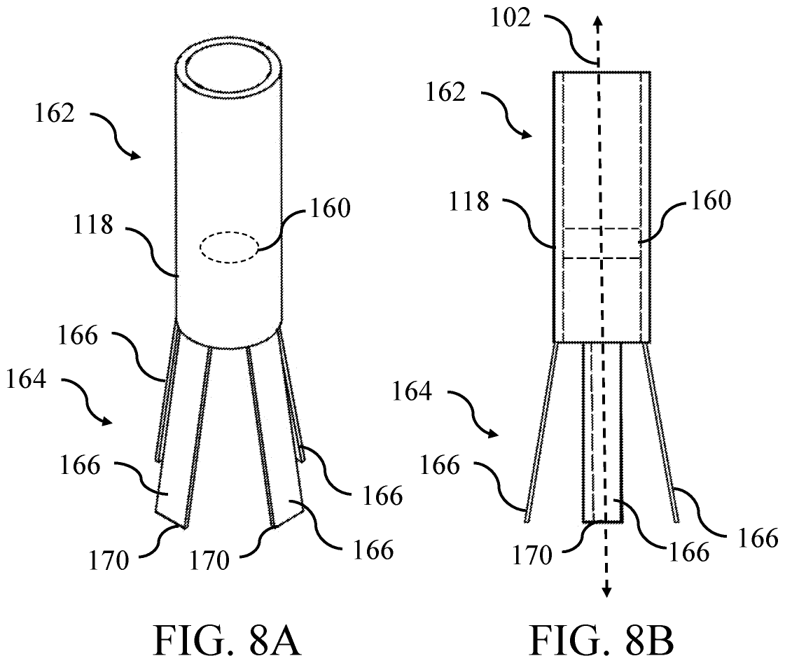

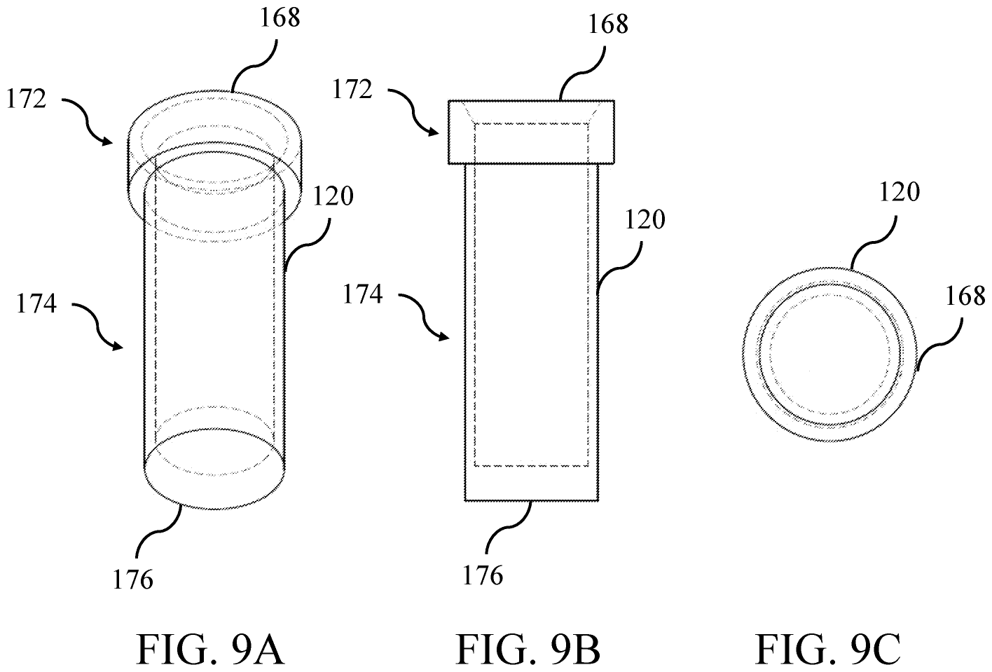

FIGS. 8A-8B show an exemplary embodiment of the firing member (118). The firing member (118) is positioned within the main shaft (116) and defines a distal portion (162) and a proximal portion (164). The distal portion (162) includes a cylindrical shape that is configured to accept the syringe holder (120) therein (see, FIGS. 9A-9C). Further, the proximal portion (164) may be fluted (i.e., the fluted end defines a radius larger than the radius of the opposing end) and define a plurality of legs (166). The legs (166) are configured to hold into place the head (168) of the syringe holder (120) (see, FIGS. 9A-9C) via a press fit coupling. In some embodiments, the firing member (118) is formed of a more rigid material than the other components, for example, a metal or brass material. Particularly, the legs (166) are capable of flexing inward and outwardly relative to the central longitudinal axis (102). As the syringe holder (120) is press fit into the legs (166), the legs (166) extend away from the longitudinal axis (102). The firing member (118) is held into place relative to the main shaft (116) by way of the proximal tips (170) of the legs being expanded outwardly from the longitudinal axis (102) such that the proximal tips (170) rest on the ridge (142) defined on the inner surface of the main shaft (116). As the firing member (118) is positioned inside the main shaft (116), with the spring (112) in place and resting against the spring compression member (110), the proximal end of the spring (112) extends through the firing member and applies longitudinal pressure against the distal end of the syringe holder (120). The firing member (118) may further include an interior pin (160) which functions as a landing or physical barrier for the spring (112) and separates the spring (112) from the syringe holder (120).

During a firing, as described above, the initiation member (114) is translated proximally within the main shaft (116). Specifically, the initiation member (114) is translated proximally over top of the firing member (118) such that the proximal end of the initiation member (114) contacts the legs (166) and forces each of them inward toward the longitudinal axis (102). By doing so, and while under longitudinal pressure from the spring (112) resting against the distal end of the syringe holder (120), the legs (166) release from the ridge (142) and the spring (112) forces the firing member (118), syringe holder (120), and syringe (122) proximally. As the syringe holder (120) meets the cap (128), proximal translation is stopped and the remaining force causes the plunger of the syringe (122) to decompress and deliver the medicament.

FIGS. 9A-9C show an exemplary embodiment of the syringe holder (120). As described, the syringe holder (120) includes a distal portion (174) having a first circumference and a proximal head portion (172) having a second circumference that is larger than first circumference. The syringe holder (120) is configured to accept the syringe (122) and needle (124) combination therein, including the moveable portions containing the vial of treatment. The distal end (176) is configured to abut the spring (112).

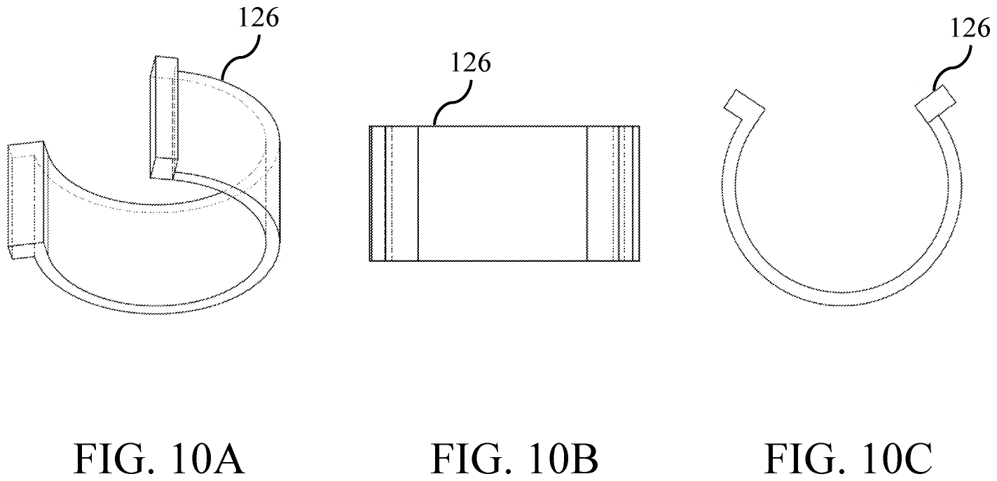

FIGS. 10A-10C show an exemplary embodiment of the safety clip (126). The safety clip (126) is configured to removably clip around the proximal end of the apparatus (100), and specifically around the main shaft (116) (see, FIG. 1) between the outer shroud (108) and the cap (128). As the safety clip (126) is positioned around the main shaft (116), the outer shroud (108) is incapable being translated proximally toward the cap (128) relative to the main shaft (116), therefore a firing of the syringe (122) and needle (124) is prevented while the apparatus (100) is not in use.

FIGS. 11A-11C show an exemplary embodiment of the cap (128). The cap (128) is configured to couple with the proximal end (154) of the main shaft (116). Particularly, the plurality of protrusions (178) of the cap (128) are configured to insert into the plurality of slots (146) defined by the main shaft (116) (see, FIGS. 4A-4B). The cap (128) operates to close in the proximal end to protect the components therein. Further, the cap (128) may include a central opening (180) whereby the needle (124) is configured to pass through during use.

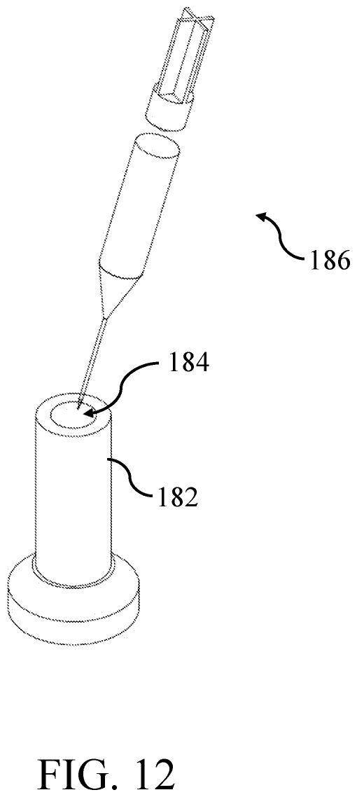

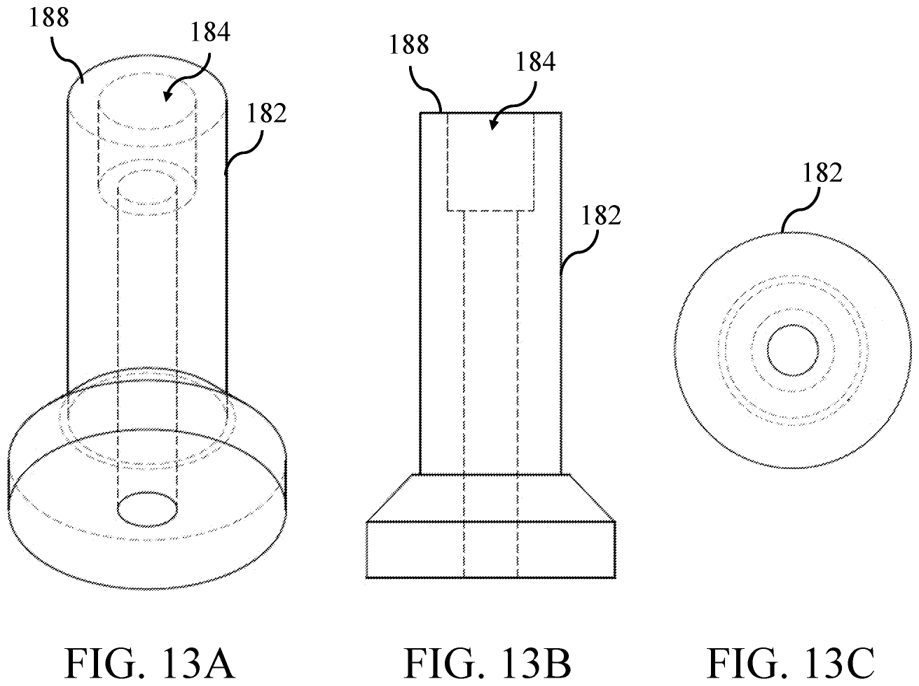

Once a treatment is administered from the apparatus (100), the syringe (122) (i.e., the treatment medicine contained therein) may be replaced before a subsequent use. As such, as shown in FIG. 12, the apparatus (100) can optionally include a stand apparatus (182) that is operable to replace the syringe (122) and reset the firing mechanism described above. With reference to FIGS. 13A-13C, showing one exemplary stand apparatus (182), a user may begin the syringe (122) replacement and resetting procedure by removing the cap (128) and removing the outer shroud (108). The outer shroud (108) can be twisted such that the protrusions (134) move along the U-shaped slots (132) of the main shaft (116). Once the outer shroud (108) meets the hard stop, the protrusions (134) can be moved up the second leg (138) for full removal. Next, the user may manually pull the spent syringe (122) and needle (124) combination from the apparatus (100). Thereafter, the user can position a replacement new syringe and needle combination (186) into the opening (184) of the stand apparatus (182). The user can then align the open proximal end with the new syringe and needle combination (186) and press the apparatus (100) downward toward the stand apparatus (100). By doing so, the new syringe and needle combination (186) will press-fit into the syringe holder (120) and remain in place. Further, as the top edge (188) of the stand apparatus (182) inserts into the open proximal end, the top edge (188) will contact the proximal end of the syringe holder (120) to translate it and the firing member (118) distally within the main shaft (116). As such, the legs (166) are moved back into their initial positions resting against the ridge (142) and awaiting a firing initiation from the initiation member (114). Finally, the user can replace the cap (128), outer shroud (108), and safety clip (126) until the next use is required.

Reference systems that may be used herein can refer generally to various directions (for example, upper, lower, forward and rearward), which are merely offered to assist the reader in understanding the various embodiments of the disclosure and are not to be interpreted as limiting. Other reference systems may be used to describe various embodiments, such as those where directions are referenced to the portions of the device, for example, toward or away from a particular element, or in relations to the structure generally (for example, inwardly or outwardly).

While examples, one or more representative embodiments and specific forms of the disclosure have been illustrated and described in detail in the drawings and foregoing description, the same is to be considered as illustrative and not restrictive or limiting. The description of particular features in one embodiment does not imply that those particular features are necessarily limited to that one embodiment. Some or all of the features of one embodiment can be used in combination with some or all of the features of other embodiments as would be understood by one of ordinary skill in the art, whether or not explicitly described as such. One or more exemplary embodiments have been shown and described, and all changes and modifications that come within the spirit of the disclosure are desired to be protected.

I/we claim:

1. An auto-injector, comprising:
(a) a housing having a central cavity, opposing ends, and a longitudinal axis defined spanning between opposing ends, wherein the housing includes at least one first protrusion extending toward the central cavity from an inward facing surface of the housing;
(b) an actuation assembly shaped to position within the central cavity of the housing, wherein the actuation assembly defines at least one first slot accessible from an interior surface of the actuation assembly and at least one second slot accessible from an exterior surface of the actuation assembly, wherein the at least one first protrusion of the housing is configured to align with and translate within the at least one second slot, wherein the actuation assembly is configured to at least partially internally house:
(i) a compression spring;
(ii) a spring compression member configured to couple with the actuation assembly and contact the compression spring, wherein the spring compression member includes at least one second protrusion configured to translate within the at least one first slot of the actuation assembly;
(iii) a needle selectively operable to eject a fluid therefrom, wherein the needle is selectively operable to extend from a proximal end of the housing;
(iv) a firing member selectively translatable between an unfired position and a fired position, wherein:
(A) while in the unfired position, the compression spring is restricted into a compressed state, wherein the compression spring in the compressed state is operable to position the needle within the central cavity of the housing; and
(B) while in the fired position, the compression spring is unrestricted into an expanded state, wherein the compression spring in the expanded state is operable to transition the needle outward from the central cavity of the housing.

2. The auto-injector of claim 1, comprising a firing initiation member configured to couple between the spring compression member and the actuation assembly and translate parallel to the longitudinal axis between a first position and a second position relative to the housing, wherein in the first position the firing member is held in the unfired position, wherein during a translation into the second position the firing member is translated into the fired position.

3. The auto-injector of claim 2, wherein a translation of the housing proximally relative to the actuation assembly is operable to translate the firing initiation member from the first position to the second position.

4. The auto-injector of claim 2, wherein the firing initiation member includes at least one third slot configured to accept the at least one second protrusion of the spring compression member.

5. The auto-injector of claim 4, wherein the firing initiation member is configured to be translated between the first position and the second position via the at least one first protrusion through the at least one third slot.

6. The auto-injector of claim 1, comprising a syringe, wherein the syringe includes the needle fluidly coupled with a fluid reservoir.

7. The auto-injector of claim 6, comprising a syringe holder configured to receive the syringe at least partially within, wherein the syringe holder includes a body portion and a head portion, wherein the firing member is configured to accept the body portion of the syringe holder at least partially within.

8. The auto-injector of claim 1, wherein the actuation assembly includes a raised ridge around the internal surface, wherein the firing member defines a cylindrical shape having a first radius at a first end and a second radius at a second end, wherein the second radius is larger than the first radius, wherein the second end of the firing member is formed by a plurality of legs , wherein each leg of the plurality of legs is configured to lodge against the raised ridge while the firing member is in the unfired position.

9. The auto-injector of claim 8, comprising a firing initiation member configured to couple between the spring compression member and the actuation assembly and translate parallel to the longitudinal axis between a first position and a second position relative to the housing, wherein when the firing member translates from the unfired position to the fired position, the firing initiation member is configured to translate over each of the plurality of legs to thereby decrease the second radius of the firing member and dislodge each leg of the plurality of legs from the raised ridge.

10. The auto-injector of claim 1, comprising an end cap configured to removably couple with the actuation assembly at an end of the actuation assembly adjacent to the needle.

11. The auto-injector of claim 10, wherein the needle is configured to pass through the end cap while the firing member is in the fired position.

12. The auto-injector of claim 1, wherein the housing and actuation assembly are restricted from being uncoupled while the firing member is in the unfired position, wherein the housing and actuation assembly are able to be uncoupled while the firing member is in the fired position.

13. The auto-injector of claim 1, comprising a safety clip removably positioned around a proximal end of the actuation assembly, wherein while the safety clip is positioned around a proximal end of the actuation assembly the housing is restricted from translating proximally relative to the actuation assembly, wherein while the safety clip is removed from the proximal end of the actuation assembly the housing is permitted to translate proximally relative to the actuation assembly.

14. An auto-injector, comprising:
(a) a housing having a central cavity, opposing ends, and a longitudinal axis defined spanning between the opposing ends;
(b) an actuation assembly shaped to position within the central cavity of the housing, wherein the actuation assembly is configured to at least partially internally house:
(i) a spring;
(ii) a spring compression member configured to couple with the actuation assembly and contact the spring;
(iii) a needle selectively operable to eject a fluid therefrom;
(iv) a firing member selectively translatable between an unfired position and a fired position, wherein:
(A) while in the unfired position, the needle is held within the central cavity of the housing; and
(B) while in the fired position, the needle is extended outward from the central cavity of the housing; and
(v) a firing initiation member configured to couple between the spring compression member and the actuation assembly and translate parallel to the longitudinal axis between a first position and a second position relative to the housing, wherein in the first position the firing member is held in the unfired position, wherein during a translation into the second position the firing member is translated into the fired position.

15. The auto-injector of claim 14, wherein a translation of the housing proximally relative to the actuation assembly is operable to translate the firing initiation member from the first position to the second position.

16. The auto-injector of claim 14, comprising an end cap configured to removably couple with the actuation assembly at an end of the actuation assembly adjacent to the needle, wherein the needle is configured to pass through the end cap while the firing member is in the fired position.

17. The auto-injector of claim 14, wherein the housing and actuation assembly are restricted from being uncoupled while the firing member is in the unfired position, wherein the housing and actuation assembly are able to be uncoupled while the firing member is in the fired position.

18. A method of resetting an auto-injector, wherein the auto-injector includes a housing and an actuation assembly shaped to position within the housing, wherein the actuation assembly is configured to at least partially internally house a spring, a spring compression member configured to couple with the actuation assembly and contact the spring, a syringe holder, and a firing member selectively translatable between an unfired position whereby a needle of a syringe is held within the housing and a fired position whereby the needle of the syringe is extended proximally outward from the housing, wherein the actuation assembly includes a raised ridge around an internal surface, wherein the firing member defines a first radius at a first end and a second radius at a second end, wherein the second radius is larger than the first radius, wherein the second end of the firing member is formed by a plurality of legs, the method comprising:
(a) inserting an unspent syringe into the syringe holder;
(b) applying a distal force to the syringe holder thereby driving the firing member distally; and
(c) positioning the firing member into the unfired position, wherein in the unfired position each leg of the plurality of legs is lodged against the raised ridge of the actuation assembly.

19. The method of claim 18, wherein the auto-injector includes a cap configured to couple with a proximal end of the housing, the method comprising:
prior to inserting the unspent syringe into the syringe holder, removing the cap.

20. The method of claim 18, wherein the auto-injector includes a firing initiation member configured to couple between the spring compression member and the actuation assembly and translate between a first position and a second position relative to the housing, wherein applying a distal force to the syringe holder translates the firing initiation member distally.
